(19) United States
(12) Patent Application Publication
(10) Pub. No.: US 2004/0098034 A1
(43) Pub. Date: May 20, 2004

(54) Femoral arterial/venous haemostasis device

(76) Inventor: Brian Paul O'Connor, Horsley NSW (AU)

Correspondence Address: MOLINS AND CO, LEVEL 25 CHIFLEY TOWER, SYDNEY NSW, 2000, AU

(21) Appl. No.: 10/450,193

(22) PCT Filed: Dec. 13, 2001
(86) PCT No.: PCT/AU01/01624

(30) Foreign Application Priority Data
Dec. 13, 2000 (AU) PR-2040

(51) Int. Cl. A61B 17/00

(52) U.S. Cl. 606/201

(57) ABSTRACT

A femoral arterial and/or venous haemostasis device is disclosed. The device is a femoral haemostasis clamp (1), it allows direct pressure to be exerted onto the femoral artery and/or vein when removing femoral sheaths from post diagnostic or interventional surgery. The clamp includes a base plate (2) for providing a first clamping surface for the limb of a patient; a support post (4) extending upwardly from the base; an arm (5) extending between a first end mounted to the post and a second end distal from the post; and a pressure pad (13) having a notch at one end to accommodate either an arterial and/or venous sheath, the pressure pad (13) being displaceable relative to the second end and to the base for moving the surfaces into and out of clamping engagement with the limb. Preferably at the second end of the arm a rod (29) is slidably and rotatably mounted by means of a ratchet mechanism, the pad being fixedly attached to an end of the rod.

The present invention relates to a haemostasis pressure pad and in particular to a haemostasis pressure pad for a haemostasis clamp. The invention has been developed primarily for invasive cardiac diagnostic surgery and will be described hereinafter with reference to that application. However, it will be appreciated that the invention is not limited to that particular field of use.

### BACKGROUND ART

It has been known that applying pressure to a wound in a patient will promote haemostasis, that is, the application of pressure stops or slows bleeding of the wound. This technique of promoting adequate haemostasis has long been used in the medical field and in surgical procedures. One such procedure in which the technique is often used is during invasive cardiac diagnostic surgery and treatment In this procedure a catheter and sheath is inserted into an artery and/or vein at a puncture site in a limb of the patient. At the conclusion of the procedure, pressure is applied at the puncture site to facilitate the removal of the sheaths and achieve adequate haemostasis. While pressure can be applied directly by a skilled hand, to do so means that the hand cannot attend to other duties for a significant period of time. To overcome this problem artery clamps and pressure pads to directly apply pressure to the catheter entry site have been developed. One type of pad is a simple disc, which is generally made from plastic and which is held in place by a stand or cap to enable the nurse or other operator to safely and quickly remove the femoral sheath or sheaths. However, the simple round design of there pads makes correct alignment of the disc with the arterial sheath difficult.

In partial answer to this problem, it has been know to include a narrow alignment notch in the disc to allow visualisation of the puncture site and thus enable alignment However, these discs are only designed for use when removing an arterial sheath only. Patients often have an arterial and venous sheath that need to be removed.

Another problem of the current disc relates to its small size, which minimises the dispersal of pressure and increases patient discomfort throughout the procedure. Patients have commented in recent surveys that, “the sheath removal part is the most discomforting part of the whole interventional cardiac procedure . . . ”. Also, the small size of disc means that incorrect disc positioning can place pressure on the femoral nerve that runs along side the femoral artery and vein, which can cause a life-threatening situation called a “vaso vagal”. The flow of blood through and the pulse pressure wave play a vital part in the formation and the hardening of the dot to seal the hole. The smaller size disc needs more pressure to be applied to reduce blood flow and allow dot formation. The tolerance of maintaining a comfortable position decreases with more pressure being applied. The risk of femoral complications occuring increases due to the patients discomfort and the likelihood the patient will move instinctively to improve their comfort.

The current design of clamp consists of a base, a vertical pole extending upwardly and a horizontal arm vertically extending to the pole and having a free end for supporting the pad. The horizontal arm can be locked by way of friction cam lock acting on the pole. As the operator gradually removes the sheath their aim is to then apply pressure by pushing down the horizontal arm. When the operator releases pressure from the horizontal arm, the arm locks against the vertical pole and pressure is maintained on the site.

To achieve haemostasis, it is paramount to properly set up the device. If incorrect, the risk of vascular complications, patient trauma, and the overall costs of the procedure increase. It is also known that an improperly set device will give rise to a pressure imbalance across the disk surface and the femoral artery.

The further the disc is away from the vertical pole, the greater the probability of pressure imbalance and the risk of vascular complications. The problem is exacerbated by the fact that the arm is made from plastic and able to flex. This flexing often results in a rotation of the face of the disc with respect to the site-giving rise to a pressure imbalance and/or loss of pressure. Additionally, the size of the patient, the correct positioning of the of the disk prior to sheath removal, and the movement of the patient after sheath removal all increase the risk of the horizontal arm flexing, disc slippage and as a result, loss of pressure over the femoral artery. Correct set-up of the current device is also made difficult because, once the sheath is removed, the operator must hold the horizontal arm still and in position, and then, with the other hand, tighten the locking nut to stop the arm from slipping up. The two operations can be difficult to coordinate. Moreover, if the patient continues to bleed, repositioning of the clamp will be required.

### SUMMARY OF THE INVENTION

It is an object of the present invention to overcome or ameliorate at least one of the disadvantages of the prior art, or to provide a useful alternative. According to a first aspect, the invention provides a haemostasis pressure pad for a haemostasis clamp, the pad including: a body having a substantially planar elongate engagement surface for abutting a limb of a patient; and a pair of extensions projecting from the body to respective free ends, the extensions forming respective continuations of the planar surface and defining an intermediate notch having its largest dimension at the free ends.

Preferably the body includes a second surface, which meets the engagement surface at the periphery of the pad and which, at least in part, extends along a substantially elliptical path having a major axis and a minor axis.

Preferably the notch is large enough to allow visualisation of at least two proximate puncture sites in the limb.

Preferably the engagement surface is etched to provide a textured, substantially non-slip surface.

Preferably the pad has a centrally located mounting boss with a hexagonal aperture for receiving the end of a locating rod.

The pad further preferably includes strengthening ribs radiating from the mounting boss towards the periphery for resisting deformation of the first surface.

According to another aspect, the invention provides a haemostasis clamp for clamping the limb of a patient, the clamp including:

a base for providing a first clamping surface for the limb;

a support post extending upwardly from said base; an arm extending between a first end mounted to the post and a second end distal from the post; and a pressure pad mounted to the second end and including a second clamping surface, the pad being displaceable relative to the second end and to the base for moving the surfaces into and out of clamping engagement with the limb. Preferably the arm is attached to the post at the first end by a releasable clamp and at the second end of the arm, a rod is slidably and rotatably mounted by means of a ratchet mechanism. In turn, the pad is fixedly attached to the other end of the rod.

### BRIEF DESCRIPTION OF THE DRAWINGS

Preferred embodiments of the invention will now be described by way of example only, with reference to the accompanying drawings, in which:

FIG. 1 is a plan view of a pressure pad according to the present invention;

FIG. 2 is a side elevation of the pressure pad of FIG. 1;

FIG. 3 is a perspective view from above of the pressure pad of FIG. 1;

FIG. 4 is a perspective view of a haemostasis clamp according to the present invention;

FIG. 5 is an enlarged side elevation view of a portion of the amp of FIG. 4;

FIG. 6 is a plan view of the portion of the clamp depicted in FIG. 5;

FIG. 7 is an enlarged side elevation of a portion of the clamp of FIG. 4;

FIG. 8 is a top and side view of an alternative embodiment of the arm; and

FIG. 9 is an exploded view of the rod, handle and pad, with a detail of the rod.

### PREFERRED MODE OF CARRYING OUT THE INVENTION

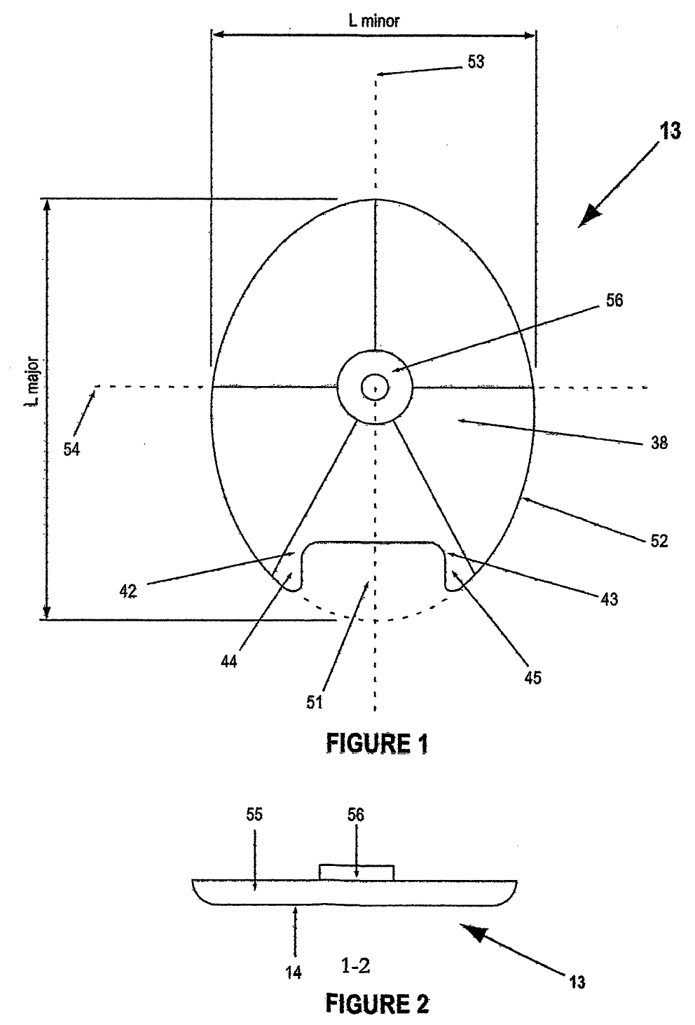

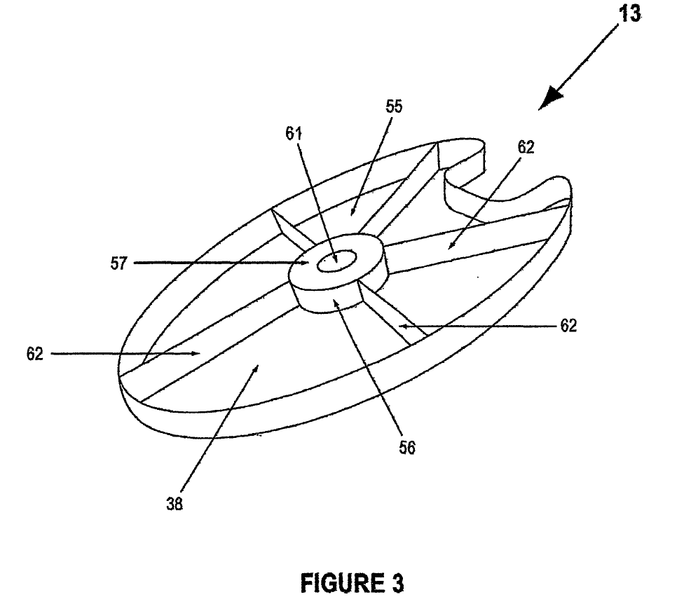

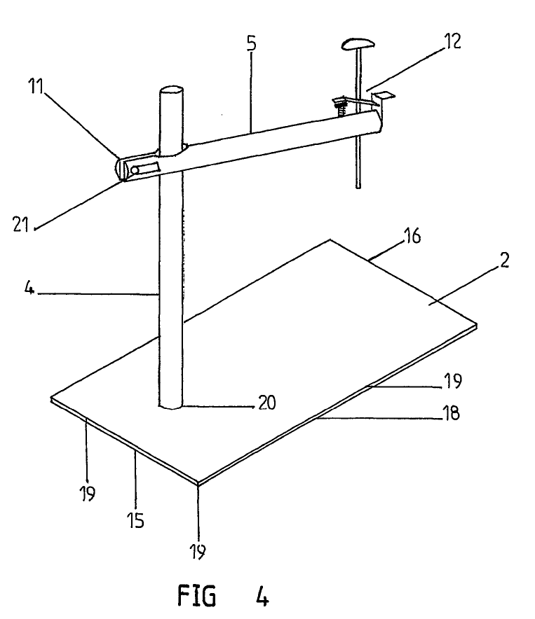

Referring to the drawings, and in particular to FIG. 4, there is illustrated a haemostasis clamp 1 for clamping the limb of a patient (not shown). The clamp includes a generally rectangular base 2 for providing a substantially planar upwardly facing clamping surface 3 for the limb. A support post 4 of circular cross section extends upwardly from base 2, and an arm 5 extends between a first end 11 mounted to the post and a second end 12 distal from the post. A pressure pad 13, as shown in FIGS. 1 to 3, is mounted to end 12 and includes a downwardly facing clamping surface 14. The pad 13 is displaceable relative to end 12 and base 2 for moving surfaces 3 and 14 into and out of clamping engagement with the limb.

Base 2 is fashioned from mild steel plate and is of sufficient weight to promote stabilisation of the clamp when in use. Further, base 2 includes two transverse sides 15 and 16, and two longitudinal sides 17 and 18 each having filleted or chamfered edges 19 to reduce the probability of injury to patients and users.

Clamping surface 3 includes a substantially non-slip section which, in this embodiment, is a substantially planar rubberised mat. However, in other embodiments the mat is textured or roughened to enhance the non-slip function.

Base 2 includes a mounting boss 20 which is disposed adjacent side 15 and equidistant from longitudinal sides 17 and 18 for threadedly receiving one end of post 4. This allows for convenient disassembly of clamp 1. In further embodiments, support 4 is fixedly attached to base 2 by welding, riveting, an interference fit or similar permanent attachment

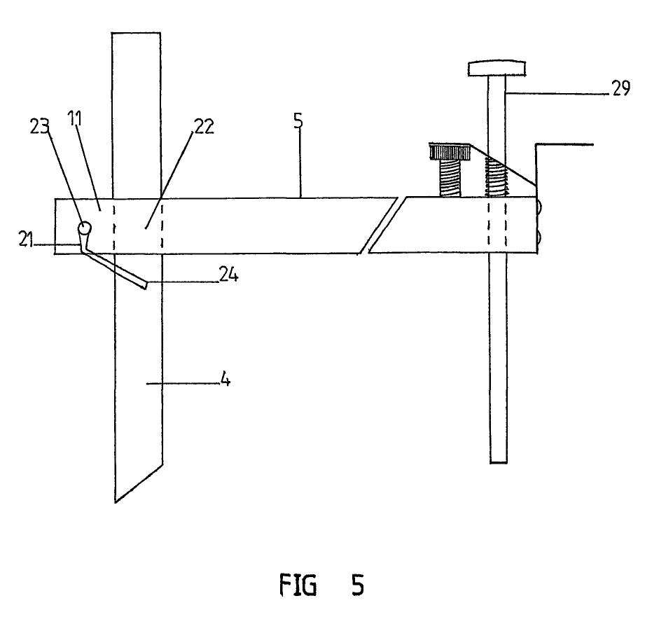

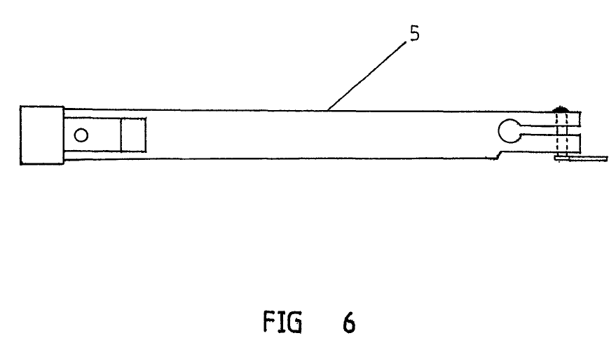

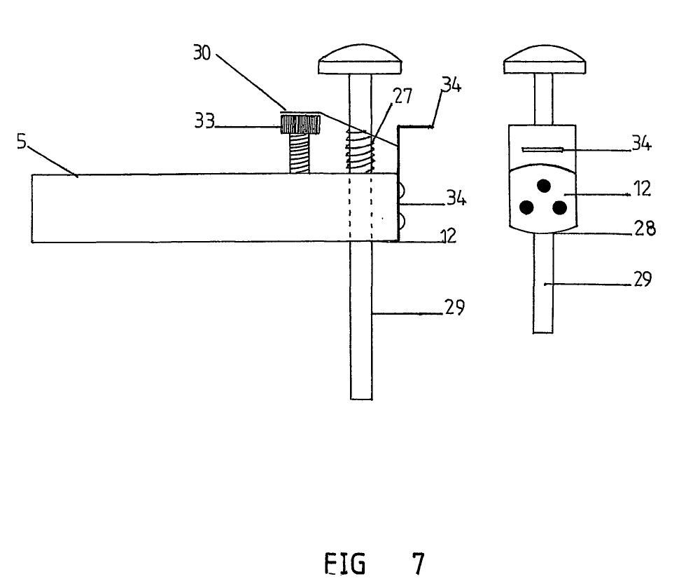

End 11 is slidably and rotatably mounted to post 4 by a releasable clamp 21; as best shown in FIGS. 4 and 5. Clamp 21 allows arm 5 to be selectively lockingly retained at one of an infinite number of relative positions along post 4. Clamp 21 includes a collar 22, which surrounds and receives post 4. A locking screw 23 is threadedly attached to clamp 21 and when tightened acts against post 4 to lock arm 5 in position A handle 24 is provided for activation of screw 23.

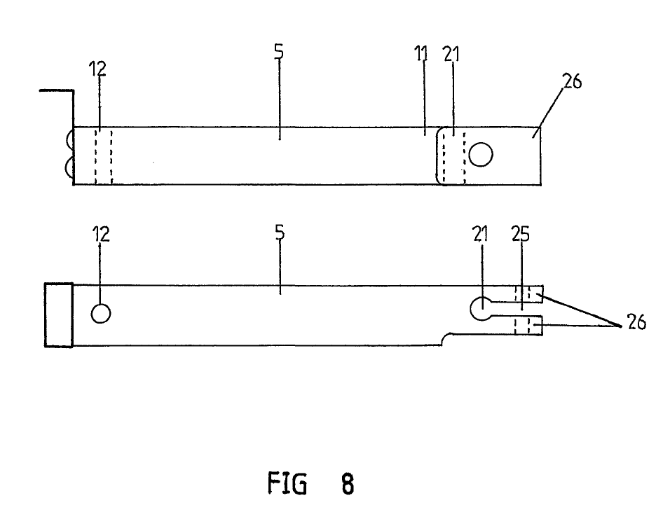

In other embodiments clamp 21 is a releasable compression clamp, as best shown in FIG. 8 where corresponding features are denoted by corresponding reference numerals. The clamp includes a collar 25 split into two opposing sides 26 which collectively surround post 4. A quick release cam locking is attached to the sides 27 and configured so that when tightened, the sides are drawn together in releasable clamping engagement with post 4. A handle is provided for activation of the locking device.

In other embodiments, post 4 includes a plurality of discretely spaced apart locking formations for providing a corresponding plurality of locking positions relative to base 2. Arm 5 is a stainless steel tube of similar length to base 2 and includes a ratchet mechanism 26 at end 11. In other embodiments arm 5 is of a polymer based material or of non uniform cross section.

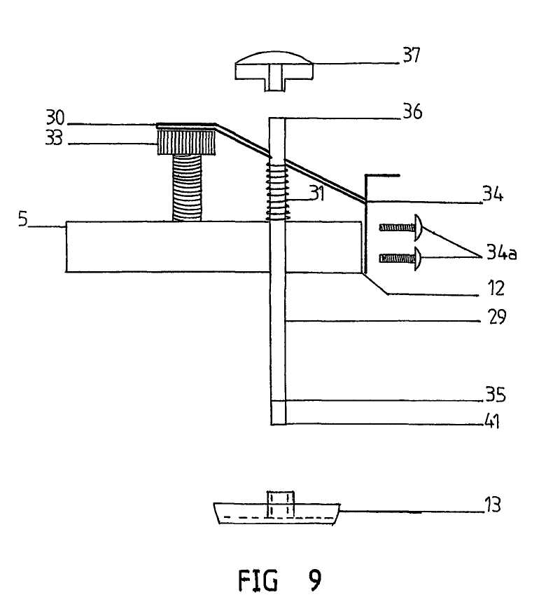

Ratchet 27 includes an aperture 28 which extends parallel to post 4 for receiving a rod 29. Ratchet 27 further includes a lock plate 30 which is mounted to grip plate 34 for horizontal movement with respect to rod 29. Lock plate 30 resiliently biased into engagement with the rod by the offset angle of lock plate 30 resting on the compression coil spring 31. Lock plate 30 can be secured in its position by adjusting the height of lock screw 33. Lock screw 33 threads into post 5. Lock screw 33 allows the operator to lock the position of lock plate 30 to stop the disengagement of rod 29 from lock plate 30 and the loss of pressure at end 14.Depressing lock plate 30 allows rod 29 to freely travel through post 5 in both directions. The lock plate 30 is returned to its original position by the compression coil spring 31 when lock plate 30 is released. Rod 29 automatically engages again against lock plate 30. Rod 29 can only move in one direction through post 5 and will maintain the clamping surfaces 3 and 14 in a fixed spaced apart relationship. This is best seen in FIG. 9. This allows rod 29 to be moved toward, but not away from, surface 3. Lock plate 30 is manually moved out of engagement with rod 29 to allow the rod to be moved in the opposite direction Rod 29 is stainless steel and extends between a lower end 35, nearer surface 3 and 5 an upper end 36, as best shown in FIG. 9.

End 35 may include a hexagonal formation 41 for engaging pad 13 in a fixed rotational relationship. End 36 includes a handle 37 for allowing manual lowering and raising of rod 29. The handle is in the form of a contoured circular disc to complement the palm of the users hand when applying downward pressure. At the end 12 the grip plate 34 allows the operator to ergonomically rest their palm of their hand on handle 37 and place fingers under grip plate 34. Grip plate 34 allows greater control of rod 29 when applying and releasing the pressure being applied. Grip plate 34 is of stainless steel construction secured to end 12 by three dome allen key threaded screws 34a.

Pad 13 includes a plastics body 38 having a substantially planar elongate engagement surface defined by surface 14 for abutting a limb of the patient Pad 13 further includes a pair of integral extensions 42 and 43 which project from body 38 to respective free ends 44 and 45. These extensions 42 and 43 form respective continuations of surface 14 and define an intermediate notch 51 which has its largest dimension at ends 44 and 45. Pad 13 has a periphery 52 which at least in part extends along a substantially elliptical path having a major axis 53 and a minor axis 54. In this embodiment the width L_{Minor} of the pad on the minor axis 54 is approximately 50 mm and the length Lₘₐⱼₒᵣ of the pad on the major axis 53 is approximately 70 mm. However in other embodiments the width Lₘᵢₙₒᵣ is between 50% and 90% of that of length Lₘₐⱼₒᵣ The width of notch 51 at ends 44 and 45 of extensions 42 and 43 is approximately 15 mm and the depth of notch 51 is approximately 10 mm. Thus, notch 51 is of sufficient dimension to allow visualisation of at least two proximate puncture sites in the limb. Surface 14 includes a textured finish to facilitate non-slip abutment with the limb.

Body 38 also includes an opposite surface 55, which meets surface 14 at a periphery in the form of a rounded lip 56. The lip provides surface 14 with an edge devoid of sharp corners. Surface 55 includes mounting means, in the form of a centrally located mounting boss 57, for mounting pad 13 to formation 41. Boss 57 includes a hexagonal locating aperture 61 for complementarily receiving end 35 of rod 29 in fixed rotational engagement

Surface 53 further includes five radially diverging strengthening ribs 62 for resisting deformation of surface 14. Ribs 62 radiate from boss 55 and terminate at periphery 52. In other embodiments a different number and or configuration of ribs are used. Moreover, in still further embodiments, pad 13 does not include ribs and gains sufficienty rigidity to resist the deformation commonly encountered by other means. For example, through the use of thicker and stiffer plastic material.

Clamp 1 is designed for any procedure where a catheter and sheath have been inserted into the artery and/or vein of a patient. During removal of the sheath, clamp 1 is applied to the limb so that surface 14 abuts the limb at or adjacent to the entry or puncture site. Once the sheath is removed surface 14 applies adequate pressure to promote haemostasis. The clamp is suitable for use in conjunction with either Femoral or Brachial sheaths. Surface 14 includes a large area for ensuring that significant portions of the arterial and/or venous vessels are compressed thus ensuring adequate haemostasis to allow clot formation. This also minimises the risk of both haematoma development and generalised bruising at that site.

In use, the base 2 is positioned between the patient (not shown) and the bottom of the mattress (not shown). The weight of the patient assists in the stabilisation of clamp 1 Prior to a clamping operation; pad 13 is mounted to end 35 of rod 29. The rod is positioned so that pad 13 is immediately adjacent to end 12 of arm 5. Arm 5 is then moved relative to support post 4 both vertically and horizontally until pad 13 is appropriately positioned above the femoral artery at the puncture site. Notch 51 is used to allow ready visualisation of the catheter puncture site before pad 13 touches the limb. Prior to removal of the catheter sheets, following cardiac surgery for example, rod 29 is lowered until surface 14 comes into contact with the limb of the patient. As the catheter or catheters are removed, pad 13 is gradually progressed downwardly, to apply controlled pressure at the puncture site. Ratchet mechanism 27 allows pressure to be applied and maintained with one hand so that pad 13 remains under pressure until relieved.

Once the pressure has been applied for sufficient length of time to promote haemostasis, the pressure of pad 13 on the limb is gradually released by manually extracting pin 30 from engagement with rod 29 in gradual increments every five minutes over a 20-40 minute period. If bleeding or swelling is detected pad 13 can be easily re applied by ratchet mechanism 27. The process is repeated until pad 13 has reached its starting position. Arm 5 and pad 13 are then moved upward, out of clamping engagement, and clamp 1 removed from under the buttock of the patient. Manual pressure is applied proximal to pad 13 prior to removing Arm 5 and pad 13 from its clamping engagement The pad, being disposable, is discarded. Clamp 1 is then sterilised in an appropriate manner and provided with a new sterilised pad equipped for re-use.

The major advantages of the pad of the preferred embodiments are:

the pad provides for removal of more than one catheters by having a wide notch the pad has a large surface area which distributes the load over a larger area to reduce the risk of vascular complications and improve patient comfort; and the shape and size of the pad compresses a greater number of vessels, slowing blood flow and dispersing the arterial pulse wave.

The clamp of the preferred embodiment has the major advantages of.

Having adjustment at the clamping end of the arm as well as the support end to allow accurate control of the pressure applied at the puncture site; providing automatic locking of the rod and pad via a ratchet mechanism at the pad end of arm to allow one handed operation once the clamp is in place; having a rigid arm which has negligible flex under normal working loads for minimising pad slippage; allowing direct pressure to be gradually applied to the artery and/or vein; and allowing pressure to be gradually released, as required.

Although the invention has been described with reference to specific examples it will be appreciated by those skilled in the art that the invention may be embodied in many other forms.

The claims defining the invention are as follows:

1. A femoral arterial/ and or venous sheath haemostasis device that allows the operator to apply direct pressure proximal to entry points of sheath/s while simultaneously removing the femoral sheath/s.

2. The pressure is dispersed through a dear pressure pad that has a wide notch at one end accommodate either an arterial and/or venous sheath.

3. The pressure pad can actively travel only in one direction. Once resistance is place on the pressure pad the clamp locks at its current position against the lock plate

4. The pressure pad position is secured by fixating the horizontal arm against the vertical pole.

5. The gradual application of pressure while the sheaths are removed allows the operator better control of the pressure being applied.

6. Once the desired pressure has been reached the clamp will automatically lock maintaining pressure to stop blood flow through the femoral artery and/or vein.

7. A lock screw secures the clamp in its position to stop accidental activation of the lock plate and loss of pressure.

8. The lock plate when activated allows free travel of the pressure pad in both directions. The resistance allows the pressure pad rise slightly easing pressure and slowly restoring blood flow to the distal limb.

9. The lock plate is returned to its original position by a compression spring again locking the clamp at the desired pressure.

10. The pressure pad is a large oval size to fit ergonomically into the groin area.

11. It is longer from proximal to distal end to allow more compression of the femoral vessels and reduce the pulse wave when blood flow is restored.
